(19) United States
(12) Patent Application Publication
(10) Pub. No.: US 2008/0241260 A1
(43) Pub. Date: Oct. 2, 2008

(54) Compositions for Enhanced Absorption of Biologically Active Agents

(76) Inventors: Padma Venkitachalam Devarajan, Mumbai (IN); Ganeshchandra Shivajirao Sonavane, Maharashtra (IN); Hetal Jayantilal Gala, Mumbai (IN)

Correspondence Address: Pearl Cohen Zedek Latzer, LLP, 1500 Broadway, 12th Floor, New York, NY, 10036, US

(21) Appl. No.: 12/088,532

(22) PCT Filed: Sep. 28, 2005
(86) PCT No.: PCT/IN2005/000328

§ 371 Date: Mar. 28, 2008

(51) Int. Cl. A61K 9/14; A61K 38/02; A61K 39/00; A61K 9/12; A61K 31/7052; A61K 38/28

(52) U.S. Cl. 424/489; 514/2; 424/184.1; 514/44; 514/3; 424/45; 977/915

(57) ABSTRACT

The present invention relates to a novel pharmaceutical composition comprising polymeric nanoparticles with one or more biologically active agent/s for mucosal and or oral administration. Said polymeric nanoparticles further comprise of an agent that enhances absorption of said biologically active agent/s. The compositions are formulated as powders, sprays, suspension, freeze dried powders for reconstitution, tablets, capsules, pellets, wafers, patches, films, rods, pessaries, suppositories, aerosols, bioadhesive gels, creams.

### TECHNICAL FIELD

The present invention relates to a novel pharmaceutical composition comprising of plurality of polymeric nanoparticles made from pharmaceutically acceptable polymers and active biological active agent selected from group of proteins and peptides such as insulin, nucleic acids, oligonucleotides, vaccines and other biologically active molecules for mucosal (buccal, sublingual, nasal, pulmonary, etc.) and/or oral administration. The invention further relates to use of absorption enhancers in above composition for enhanced absorption of biological active agent/s through the mucosal membrane.

### BACKGROUND AND PRIOR ART

Medical use of protein and peptide like biologically active agents is constrained by three major drawbacks. The first is their short biological half-life, which requires, in some cases, frequent administrations. The second is the rapid degradation, which occurs, in mucosal tissues that generally cover the body cavities. Lastly, most biologically active agents from these categories are large molecules and therefore do not easily cross the intestinal epithelium. Therefore, the most common mode of administration of these agents is the parenteral route. However, apart from the inconvenience to the patients, parenteral delivery systems are also more expensive in terms of production and drug administration. There is therefore a need for an effective non-parenteral mode of administration that will provide protection against biological degradation and/or enhance its transport across mucosal barriers.

Insulin plays a central role in the regulation of carbohydrate, fat, and protein metabolism in the body. Diabetes mellitus (commonly referred to simply as diabetes) is a disease characterized by disregulation of metabolism, particularly glucose metabolism. Diabetes is a serious disease, which is becoming common especially in developing countries. About 120-140 million people worldwide suffer from this disease and the numbers are slated to double by 2025. Type I diabetes, or insulin-dependent diabetes mellitus (IDDM), usually begins in childhood. It is characterized by atrophy of the pancreatic beta cells, resulting in a decrease or cessation of insulin production, and leaving the patient dependent on exogenous insulin for survival.

The more common Type II diabetes, or non-insulin-dependent diabetes mellitus (NIDDM), generally occurs in patients older than 40 years. Although Type II diabetes often can be treated by controlling the patient's diet, administration of exogenous insulin to supplement that secreted by the patients beta cells may also prove necessary. However various therapeutic forms of insulin exhibit poor bioavailability after oral administration. This is due to poor absorption of such macromolecules across the mucosal surfaces of the stomach, intestine and colon and enzymatic degradation in the gastrointestinal (GI) tract before they can be absorbed. Consequently, these are administered by parenteral route owing to their inability to survive the GIT conditions and poor absorption properties. The necessity for daily injection causes great deal of inconvenience and discomfort to many patients and occurrence of local reactions at the injection site. In addition, there is often an abnormal non-physiological plasma concentration profile for injected insulin. This abnormal profile is undesirable and increases the risk of side effects.

Although single or multiple daily subcutaneous injections of insulin are the mainstay of insulin delivery techniques, several other methods of insulin delivery are now available or in development, including (a) continuous subcutaneous insulin infusion by a wearable infusion pump; (b) total or segmental transplantation of a pancreas; (c) transplantation of isolated islet cells; (d) implantation of a programmable insulin pump; (e) oral, nasal, rectal and transdermal mechanisms of insulin delivery; (f) administration of insulin analogues; (g) implantation of polymeric capsules which give continuous or time-pulsed release of insulin; and (h) implantation of biohybrid artificial pancreas which uses encapsulated islets. However, these techniques being invasive in nature have their own limitations and requires person skilled inn the art along with increased cost factor. Moreover, subcutaneous and pulmonary methods of insulin delivery do not currently mimic physiological insulin needs and transplantation requires risky immunosuppression.

There is therefore continuous research going on in the direction of developing new and improved non-parenteral delivery systems particularly for biologically derived proteins and peptide drugs such as insulin.

A number of strategies have been explored and continue to be explored to design potential mucosal and/oral delivery system for biologically active agents like proteins, peptides, nucleic acids and oligonucleotides.

U.S. Pat. No. 5,804,212 describes compositions for intranasal delivery comprising a plurality of bioadhesive microspheres made of starch, gelatin, dextran, collagen or albumin and active drug from peptides, such as insulin, and antigenic vaccine. The composition may additionally comprise an absorption enhancer such as non-ionic surfactants and various bile salt derivatives. Other patents which talks of polysaccharide microparticles are U.S. Pat. No. 5,679,377; U.S. Pat. No. 5,985,305. However the composition of the present invention is different.

U.S. Pat. No. 5,641,515 discloses controlled release nanoparticles formed of a biodegradable polycyanoacrylate polymer. The process requires first polymerization of monomer and then preparation of nanoparticles using various steps including use of stabilizers and enteric coating. The present invention does not require any polymerization and further the composition is different. Several other patents also reports nanoparticles and/microparticles prepared of proteins and peptides from biodegradable poly (alkyl cyano-acrylate) (See U.S. Pat. No. 4,329,332; U.S. Pat. No. 5,500,224; U.S. Pat. No. 5,641,515; U.S. Pat. No. 5,985,309, US Patent application 20040076681). However, The composition of the present invention is novel and different from those mentioned in the above patents.

U.S. Pat. No. 6,368,586 provides methods and compositions for enhancing the bioadhesive properties of polymers used in drug delivery devices by incorporating anhydrideoligome nanoparticles such as water-insoluble metal oxides, including oxides of calcium, iron, copper and zinc. In one embodiment metal oxides can be incorporated within polymers used to form or coat drug delivery devices such as microspheres, which contain a drug or diagnostic agent. The metal oxides can be provided in the form of fine dispersion of particles on the surface of a polymer that coats or forms the devices. However, it includes complex procedure such as coating to incorporate metal oxides and solvent evaporation and polymerization techniques (alternately Phase inversion technique has also been described) for preparation of nanoparticles. The teachings of this patent are useful to enhance the ability of the variety of polymers to adhere to a tissue surface such as a mucosal membrane but it cannot enhance the permeability or absorption of the microspheres or nanoparticles containing the drug delivered into the tissues. The metal oxides used are likely to affect stability of the most labile peptide drugs like insulin and therefore such compositions are not effective in the treatment of the disease.

EP-A-023,359 and EP-A-122,023 describes a powdery pharmaceutical composition for application to the nasal mucosa and methods for administration thereof. The pharmaceutical composition allows polypeptides and derivatives thereof to be effectively absorbed through the nasal mucosa. Similarly, U.S. Pat. No. 4,250,163 describes a method for administering a medicament to the nasal mucosa where the preferred composition has mucoadhesive properties.

EP-A-230,264 describes an aqueous nasal drug delivery system for vaccines containing a high molecular weight drug, a gelling agent (e.g. hydroxyethylcellulose) and in some cases other additives (e.g. surfactants, glycerol and polyethyleneglycol). None of the above patents and applications describes the use of nanoparticles/microspheres of the present invention for nasal administration.

U.S. Pat. No. 6,884,435 describes method of preparation of biodegradable microparticles comprising a polymer, such as a poly (a-hydroxy acid), a polyhydroxy butyric acid, a polycaprolactone, a polyorthoester, a polyanhydride, or a polycyanoacrylate, and a detergent with biologically active agent like oligonucleotides. The present invention relates to nanoparticles and moreover the composition is different.

U.S. Pat. No. 6,709,825 (and also other successive patents U.S. Pat. No. 6,740,491; U.S. Pat. No. 6,750,016; U.S. Pat. No. 6,759,199; U.S. Pat. No. 6,767,702; U.S. Pat. No. 6,773,884; U.S. Pat. No. 6,828,432; U.S. Pat. No. 6,878,814) provide methods of detecting a nucleic acid. The methods comprise contacting the nucleic acid with one or more types of particles having oligonucleotides attached thereto. In one embodiment of the method, the oligonucleotides are attached to nanoparticles and have sequences complementary to portions of the sequence of the nucleic acid.

The invention further provides methods of synthesizing unique nanoparticle-oligonucleotide conjugates. The composition of the present invention does not require any conjugation and moreover the composition is different.

### OBJECT

It is therefore an object of the present invention to enhance absorption of biologically active agent with an aid of pharmaceutical composition comprising of plurality of nanoparticles prepared from polymers and at least one absorption enhancer for enhanced absorption of biologically active agent.

Another object of the invention is to provide pharmaceutical composition for mucosal and/oral administration of peptides like insulin and other biologically active agents that overcomes the limitations of prior art.

Another object of the present invention is to provide pharmaceutical composition comprising of plurality of nanoparticles for mucosal/or oral administration of peptides like insulin that have enhanced bioavailability at low dose.

Yet another object of the present invention is to provide cost effective pharmaceutical composition comprising of plurality of nanoparticles for mucosal/or oral administration of biologically active drugs, which is simple to prepare and scale-up.

A further object of the present invention is to provide pharmaceutical composition comprising of plurality of nanoparticles for mucosal/or oral administration that is easy to administer.

Yet further object of the present invention is to provide pharmaceutical composition comprising of plurality of nanoparticles for mucosal/or oral administration of peptides like insulin that are stable.

### SUMMARY OF THE INVENTION

Thus according to an aspect of the present invention, there is provided a novel pharmaceutical composition of biologically active agent/s comprising of plurality of nanoparticles prepared from polymers, and at least one absorption enhancer for enhanced absorption of biologically active agent/s through mucosal or oral route.

According to one aspect of the present invention, there is provided a pharmaceutical composition containing biologically active agent wherein the biologically active agent is a member selected from the group consisting of proteins, peptides, oligonucleotides, vaccines, nucleic acids and their analogues and compatible mixtures thereof.

According to another aspect of the present invention there is provided a pharmaceutical composition of biologically active agent comprising of plurality of nanoparticles prepared from polymers and absorption enhancers, wherein the absorption enhancers are vitamins, niacinamide, cyanocobalamin, tocopherol and derivatives thereof.

According to third aspect of the present invention, there is provided a pharmaceutical composition comprising of plurality of nanoparticles prepared from polymers wherein said polymers include anionic polysaccharides, cationic natural polymers and poly anhydrides.

In yet another aspect of the present invention, there is provided a pharmaceutical composition of biologically active agent comprising of plurality of nanoparticles prepared from polymers and at least one absorption enhancer wherein said composition may be administered by mucosal and/oral route to improve absorption and thus improve bioavailability and efficacy.

In yet another aspect of the present invention, there is provided a pharmaceutical composition, which may be formulated as powders, sprays, suspensions, freeze dried powders for reconstitution, tablets, capsules, pellets, wafers, patches, films, rods, pessaries, suppositories, aerosols, bioadhesive gels, creams.

In yet another aspect of the present invention, there is provided a pharmaceutical composition, which may be formulated as powders, sprays, suspension, freeze dried powders for reconstitution, tablets, capsules, pellets, wafers, patches, films, rods, pessaries, suppositories, aerosols, bioadhesive gels, creams and may additionally comprise of pharmaceutical excipients known in art including surfactants.

### DETAILED DESCRIPTION OF THE INVENTION

The present invention focuses on composition and methods for enhanced absorption of biologically active agent/s comprising of plurality of nanoparticles prepared from polymers, and at least one absorption enhancer for enhanced absorption of biologically active agent/s through mucosal or oral route.

### DEFINITIONS

As used herein, certain terms may have the following defined meanings.

As used herein the term a “pharmaceutical composition” is intended to mean a combination of biologically active agents, at least one absorption enhancer and other pharmaceutically acceptable excipients, said excipients known in art for their application in pharmaceutical formulations.

The term “biologically active agent” refers to any agent or chemical which is pharmacologically active, and has therapeutic value.

As used herein the term “absorption enhancer” refers to any material that acts to increase the absorption or absorption across the mucosa. Whether a given compound is an “enhancer” can be determined by comparing two formulations comprising drug, with or without the enhancer, in an in vitro/in vivo or good model test and determining whether the permeability of the drug is enhanced. The enhancer should not produce any problems of chronic toxicity and should be non-irritant and/or rapidly metabolized to a normal cell constituent that does not have any significant irritant effect in-vivo.

As used herein the term “insulin” refers to human, bovine, porcine or insulin of other animal origin or can be a recombinant product.

The term “LD₅₀” refers to median lethal concentration i.e. the concentration at which 50% of rodents (rats/mice) die.

Particularly preferred polymers are hydrogels or thermoplastics, homopolymers, copolymers or blends, natural or synthetic. The more preferred bioadhesive polymer may be acidic nonnaturally occurring polymers, such as poly(acrylic)- and/or poly(methacrylic)acid (e.g., Carbopol, Carbomer), polyanhydrides, such as poly(methylvinylether/maleic anhydride)copolymer and their mixtures; enteric polymers such as Eudragits; basic amine-bearing polymers such as chitosan and its derivatives; acidic natural polysaccharides such as alginic acid, hyaluronic acid, pectin, gum tragacanth, and karaya gum and their salts such as sodium alginate; cellulose polymers such as carboxymethylcellulose (CMC), hydroxy propyl cellulose, hydroxypropyl methyl cellulose etc., and vinyl polymers such as polyvinylalcohol; polyvinypyrrolidone; or their mixtures. The preferred polymers of the present invention include cationic natural polymers such as chitosan, anionic polysaccharides such as alginic acid and poly anhydrides such as poly(methylvinylether/maleic anhydride)copolymer.

The term “biological active agent” is used to embrace any pharmacologically active agent, including proteins, peptides, hormones, polypeptides and vaccines, or components thereof, macromolecules such as nucleic acids and its analogues, enzymes, oligonucleotides etc.; for example, calcitonin, cyclosporin, insulin, follicle stimulating hormone (FSH), luteinizing hormone (LH), vasopressin and vasopressin analogs, catalase, superoxide dismutase, interleukin-II (IL2), interferon, colony stimulating factor (CSF), tumor necrosis factor (TNF) or melanocyte-stimulating hormone Also the drug can be selected from the group consisting of antibiotics, antimicrobials, antifungals, antivirals, anesthetics, anti-tumour agents, vasoconstrictors, cardiotonics, vasodilators, antiseptics, hypotensives, sedatives anti-inflammatory agents, anti-diabetics, anti-ulcers, analgesics, anti-histaminic agents, anti-allergic agents, and antitussive-expectorants and other biologically active agents known in the art may also be contained in the pharmaceutical composition of the invention. Mixtures of the biologically active agents are also contemplated.

In one embodiment, the invention employs pharmaceutical composition comprising nanoparticles with or without absorption enhancer for enhanced absorption. As used herein, “nanoparticles” refers to particles having a diameter of preferably less than 1000 nm, and more preferably between 1.0 and 500 nm. Nanoparticles include nanospheres, which are typically solid spherical nanoparticles. Nanoparticles also include nanocapsules, which are spherical nanoparticles typically having a core of a different polymer, biological active agent, or composition These nanoparticles are formed by simplified methods known in the art without use of organic solvents. The size of the particles produced is a function of the speed of stirring or homogenization conditions used with the selected mixture of ingredients. It is within the scope of the person skilled in the art to select the exact conditions for the desired nanoparticle size. The resultant nanoparticles exhibited excellent drug loading upto 95-97%, nanosize and physicochemical stability. It is known that for many peptides and proteins, amount of drug to be administered for a resultant therapeutic effect will be of the order of a few micrograms or less. This potentiation of effect may be due to the greater retention of delivery systems at the site. The nanoparticulate composition can also afford protection of the drug against degradation by enzymes.

The compositions of present invention may advantageously comprise an absorption enhancer. Whether a given compound is an “enhancer” can be determined by comparing two formulations comprising drug, with or without the enhancer, in an in vivo or good model test and determining whether the uptake of the drug is enhanced to a clinically significant degree. The enhancer should not produce any problems of chronic toxicity and should be non-irritant and/or rapidly metabolized to a normal cell constituent that does not have any significant irritant effect in-vivo.

Literature sites various enhancing materials lysophospholipids, for example lysophosphatidylcholine from egg or soy lecithin and other lysophosphatidylcholines, Acyl carnitines (e.g. palmitoyl-dl-carnitine-chloride), chelating agents (EGTA, EDTA, alginates), surface active agents (especially non-ionic materials), fatty acids and salts, tyloxapol, and biological detergents. Also agents that modify the membrane fluidity and permeability are appropriate such as amides, enamines (e.g. phenylalanine enamine of ethylacetoacetate), malonates (e.g. diethyleneoxymethylene malonate), salicylates, bile salts and analogues and fusidates. Also reported are acidic macromolecules such as hyaluronic acid. The preferred enhancers of the present invention are selected from a group of natural and safe compounds such as vitamins more preferably tocopherol, niacinamide, cyanocobalamin and their derivatives or salts. The said absorption enhancer may be added to the nanoparticles before freeze-drying or to the final drug loaded nanoparticles.

The present invention therefore provides a pharmaceutical composition comprising of nanoparticles made from polymers containing physiologically effective amount of active drug and including a material associated with each particle having the property of increasing the bioavailability of the active drug across a mucosal membrane. The drug delivery system of the invention further provides a method for oral/mucosal administration of a drug susceptible to degradation by enzymes present in the intestine, or mixture of drugs, to a patient in need.

Specific embodiments of prepared formulations of the invention include freeze-dried nanoparticles administered in the form of a powders, sprays, suspension, freeze dried powders for reconstitution, tablets, capsules, pellets, wafers, patches, films, rods, pessaries, suppositories, aerosols, bioadhesive gels, creams or any other means allowing oral or oral mucosal administration. These pharmaceutical compositions can be formulated for immediate release, pulsatile release, controlled release, extended release, delayed release, targeted release, or targeted delayed release. Also the compositions can be formulated for sublingual, buccal, gingival, nasal or rectal route. For development of these dosage forms the composition may further include other excipients such as, cryoprotectants such as lactose, trehalose etc, corrigents such as menthol, surfactants, inert diluents, stabilizers, protease inhibitors, preservatives, hydrophobic materials, hydrophilic materials, waxes, disintegrants, superdisintegrants, diluents, binders, lubricants, solubilising agents, aromatisers, flavor masking agents, sweeteners, coloring agents, plasticizers, antioxidants, etc.

### ADVANTAGES OF SAID INVENTION

The present invention has the following advantages, which are explained with the help of subsequent examples.
1. Said pharmaceutical composition can provide a patient friendly alternative to invasive drug delivery system such as injections by increasing the absorption and further bioavailability for those biologically active agents currently administered by injection.
2. The absorption enhancers described in this invention are non-irritating, safe, and non-toxic with a high LD₅₀.
3. Said absorption enhancers aid in increasing the absorption of poorly absorbable drugs.
4. Said pharmaceutical composition permits incorporation in a range of dosage forms.
5. Said pharmaceutical composition permits administration by sublingual, buccal, gingival, nasal, rectal and/oral routes.
6. Said pharmaceutical composition enables design of stable compositions of biologically active agents.
7. Said pharmaceutical composition containing absorption enhancers provide enhanced bioavailability when administered by sublingual, buccal, gingival, nasal, rectal or oral routes permitting administration of lower doses and thereby reduce the cost of the formulations especially for expensive biologically active agents.

### EXAMPLES

The following examples further describe and demonstrate embodiments within the scope of the present invention. The examples are given solely for the purpose of illustration and not to be construed as limitations of the present invention, as many variations are possible with out departing from the spirit and scope of the invention cl Example I

### Preparation of Chitosan Nanoparticles Containing Insulin and Absorption Enhancers [AE]:

Chitosan nanoparticles with insulin were prepared using initial polymer: insulin ratio [70:30] using the principle of reverse ion precipitation.

15 ml of 0.5% w/v chitosan solution in 2% v/v dilute acetic acid was taken 30 mg insulin (Signa Chemicals) was dissolved in the dilute acid and added to above polymer. solution under overhead stirring. Nanoparticles were generated by controlled precipitation using 10% w/v sodium tripolyphosphate solution in the presence of surfactant i.e. 25 mg Pluronic F 68 (BASF Ltd.). Nanoparticles were separated by ultracentrifugation followed by washing. The sediment of nanoparticles containing drug was dispersed in water and surfactant and homogenized using APV Gaulin high-pressure homogenizer. Two batches were prepared i.e. one batch with absorption enhancer by mixing aqueous solution of absorption enhancer with the homogenized nanoparticles suspensions and the second without any enhancer as shown below.
I a: a comparative example without any enhancer.
I b: with niacinamide (AE-1) as enhancer

The resultant homogenized mixtures were freeze dried to obtain insulin-containing nanoparticles with and without absorption enhancer.

The composition of the freeze dried nanoparticles prepared is given in Table 1.

TABLE 1

Quantity/100 mg
nanoparticles

| Ingredients | I a | | I b | |
|---|---|---|---|---|
| Bovine Insulin (Sigma Chemicals) | 200 | IU | 200 | IU |
| Pluronic F 68 (BASF Ltd] | 15 | mg | 15 | mg |

| | | | |
|---|---|---|---|
| AE-1 i.e. niacinamide | — | 20 | mg |

| | |
|---|---|
| Chitosan [Medium Viscosity] | q.s. 100 mg |

### Example II

### Preparation of Alginic Acid Nanoparticles Containing Insulin [A-NP]

Alginic acid nanoparticles with insulin were prepared using initial polymer: drug ratio [70:30] by controlled precipitation.

70 mg Alginic acid (Signet Co.) was dissolved in 20 ml 0.025 N sodium hydroxide. Insulin 30 mg was dissolve in the dilute sodium hydroxide and added to above polymer solution under stirring. Nanoparticles were generated by controlled precipitation using 0.025 N hydrochloric acid in the presence of surfactant i.e. 25 mg Pluronic F 68 (BASF Ltd.) Nanoparticles were separated by ultracentrifugation followed by washing of the sediment with distilled water. The sediment of nanoparticles containing drug was dispersed in water and surfactant and homogenized using APV Gaulin high-pressure homogenizer.

Two batches were prepared i.e. one batch with absorption enhancer by mixing aqueous solution of absorption enhancer with the homogenized nanoparticles suspensions and the second without any enhancer as shown below.
II a: a comparative example without any enhancer.
II b: with niacinamide (AE-1) as enhancer

The resultant homogenized mixtures were freeze dried to obtain drug-containing nanoparticles with and without absorption enhancer.

The composition of the freeze dried nanoparticles prepared is given in Table 2.

TABLE 2

| | Quantity/100 mg | | | |
|---|---|---|---|---|
| | nanoparticles | | | |
| Ingredients | II a | | II b | |
| Bovine Insulin (Sigma Chemicals) | 800 | IU | 800 | IU |
| Pluronic F 68 (BASF Ltd] | 15 | mg | 15 | mg |

| | | | |
|---|---|---|---|
| AE-1 i.e. niacinamide | — | 20 | mg |

| | |
|---|---|
| Alginic acid [PROTACID-F-120-NM-Signet Co.] | q.s. 100 mg |

### Example III

### In-Vivo Evaluation: Effect of Formulations on Serum Glucose Level in Diabetic Rat Model Protocol for Serum Glucose Level Measurement in Diabetic Rats is as Follows

The male albino Wistar rats were injected freshly prepared streptozotocin dissolved in citrate buffer (pH: 3.6). i.v. with 60 mg/kg. All formulations were given to moderately diabetic rats (Blood glucose level 350-400 mg/dl). On the day of the test, animals were fasted overnight. Water was provided freely. The rats were anaesthetized using thiopental (20 mg/kg) intraperitoneally and initial serum glucose levels were determined (n=4). Nanoparticulate formulations were dispersed in distilled water such that 10 μl contained 0.25 u of insulin. The prepared nanoparticulate formulations (10 μl dispersion/250 gm rat equivalent to 1 u/kg) were administered by sublingual route with a micropipette. Blood samples were withdrawn after 1, 2, 3 and 5 hr and serum were separated. Serum glucose level was analyzed with Autopac glucose kit based on GOD/POD method. The results are shown in table 3 and percent relative effect of insulin loaded nanoparticles with and without absorption enhancers graphically presented in FIG. 1.

TABLE 3
Percent decrease in serum glucose level after administration of Insulin solution
(Route-S.C., Dose-1 u/kg) and compositions of examples I and II
(Route-sublingual, Dose-1 u/kg).

| | | % Decrease in serum glucose level | | | |
|---|---|---|---|---|---|
| Example | Formulation | After 1 hr | After 2 hr | After 3 hr | After 5 hr |
| | Diabetic control | 2.66 ± 1.2↑* | 1.77 ± 0.56↑ | 0.39 ± 0.20 | 1.05 ± 0.56 |
| | Insulin solution | 76.62 ± 1.08 | 83.26 ± 2.56 | 66.75 + 5.39 | 38.26 ± 3.60 |
| | [pH: 3 sol] | | | | |
| Ia | C-NP | 10.86 ± 3.31 | 25.21 ± 4.89 | 47.99 ± 5.55 | 39.36 ± 6.54 |
| Ib | C-NP with AE-1 | 30.22 + 8.02 | 53.86 + 9.67 | 63.12 + 10.25 | 63.83 + 5.34 |
| IIa | A-NP | 9.82 ± 0.76 | 16.11 ± 2.37 | 17.63 ± 2.55 | 28.49 ± 6.09 |
| IIb | A-NP with AE-1 | 25.50 + 7.07 | 41.54 + 14.16 | 56.90 + 10.26 | 55.29 + 15.52 |

↑Increase in serum glucose level

As evident from Table-3, significant reduction in serum glucose level could be obtained with C-NP and A-NP nanoparticles. Addition of absorption enhancer AE-1 revealed significant initial decrease in serum glucose level with both C-NP and A-NP. Thus, in-vivo study of insulin loaded nanoparticles in diabetic rat model showed significantly enhanced antidiabetic effect at a very low dose of 1 u/Kg. Therefore, the compositions of the invention provide excellent non-invasive alternative for insulin delivery.

### Example III

### In-Vivo Evaluation: Insulin Levels in Normal Rats

### Protocol for Insulin Measurement in Normal Rats is as Follows

The male albino Wistar rats were grouped in to control and test rats (n=2). The control rats were administered distilled water whereas test rats were administered formulations (10 μl,sublingually). The blood was withdrawn from retroorbital plexus of rats at 0,1,2,3,5 hr. Plasma was separated and insulin levels were detected by ELISA method using Merkodia (Sweden) Insulin Kit. The results are represented graphically in FIG. 2.

### DESCRIPTION OF DRAWINGS

FIG. 1 represents percent relative effect of insulin loaded nanoparticles with and without absorption enhancers administered to rats by sublingual route with reference to insulin solution given by subcutaneous route in Streptozotocin -induced diabetes in rats. (Dose: 1 u/kg).

FIG. 2 illustrates plasma insulin level after administration of distilled water (control) and insulin loaded alginic acid nanoparticles with nicotinamide (IIb) by sublingual route (Dose: 1 u/kg) in normal rats.

### DISCUSSION WITH THE HELP OF DRAWINGS

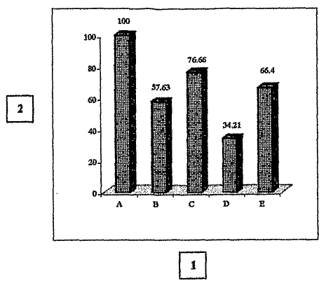

FIG. 1 represents percent relative effect of insulin loaded nanoparticles with and without absorption enhancers administered to rats by sublingual route with reference to insulin solution given by subcutaneous route in Streptozotocin-induced diabetes in rats. (Dose: 1 u/kg). Number 1 in the FIG. 1 indicates different formulations and Number 2 shows percent relative effect with respective to insulin (sublingual, 1 u/kg). Value of Block A i.e. subcutaneous insulin solution was considered as 100% and percent relative effect obtained from test formulations was determined. Blocks B and C represent percent relative effect of insulin loaded chitosan nanoparticles without (I a) and with AE-1 (I b) respectively. Blocks D and E represents insulin loaded alginic acid nanoparticles without (IIa) and with AE-1 (IIb)

As observed from the FIG. 1, sublingual administration of nanoparticles with the absorption enhancer AE-1, (Ib, IIb) showed significant percent relative effect with respect to subcutaneous insulin solution, at a very low dose of 1 u/kg, and significant increase in percent relative effect with respect to insulin loaded nanoparticles without absorption enhancer AE-1 (Ia, IIa) respectively.

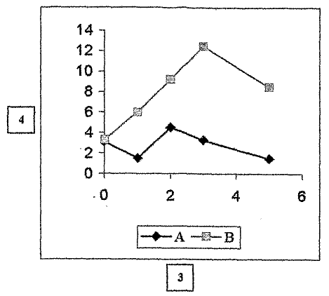

FIG. 2 illustrates plasma insulin level of control (distilled water) and insulin loaded alginic acid nanoparticles with AE-1 (IIb) administered by sublingual route in normal rats. Number 3 shows time in hours and Number 4 shows plasma insulin concentration in mcu/ml. Line represented by A shows plasma insulin level (mcu/ml) of control rats administered with distilled water, sublingually. Line B represents plasma insulin level (mcu/ml) of test rats administered with insulin loaded alginic acid nanoparticles with AE-1 (IIb). FIG. 2 confirms absorption of insulin from the nanoparticulate formulation (IIb) after sublingual administration at a very low dose of 1 u/Kg.

While the present invention is described above in connection with preferred or illustrative embodiments, these embodiments are not intended to be exhaustive or limiting of the invention. Rather, the invention is intended to cover all alternatives, modifications and equivalents included within its scope, as defined by appended claims.

1-23. (canceled)

24. A novel pharmaceutical composition of biologically active agent/s comprising of plurality of nanoparticles prepared from polymers, and at least one absorption enhancer for enhanced absorption of biologically active agent/s through mucosal or oral route.

25. A novel pharmaceutical composition according to claim 24, wherein the biologically active agent is a member selected from the group consisting of proteins, peptides, vaccines, nucleic acids and their analogues and compatible mixtures thereof.

26. A novel pharmaceutical composition according to claim 25, wherein the biologically active agent is insulin.

27. A novel pharmaceutical composition according to claim 25, wherein the biologically active agent is an oligonucleotide.

28. A novel pharmaceutical composition according to claim 24, wherein the polymer is preferably selected from among anionic polysaccharides, cationic natural polymers and polyanhydrides.

29. A novel pharmaceutical composition according to claim 28, wherein the anionic polysaccharide polymer selected is alginic acid.

30. A novel pharmaceutical composition according to claim 28, wherein the cationic polymer selected is chitosan.

31. A novel pharmaceutical composition according to claim 28, wherein the polyanhydride polymer selected is poly (methyl vinyl ether-co-maleic anhydride) and its derivatives.

32. A novel pharmaceutical composition according to claim 24, wherein the absorption enhancer comprises of vitamins and their derivatives thereof.

33. A novel pharmaceutical composition according to claim 32, wherein the absorption enhancer is preferably niacinamide, cyanocobalamin, tocopherol and/or derivatives thereof.

34. A novel pharmaceutical composition according to claim 24, wherein biologically active agent: polymer ratio by weight ranges from 1:99 to 95:5.

35. A novel pharmaceutical composition according to claim 34, wherein biologically active agent: polymer ratio by weight preferably is 10:90 to 50:50.

36. A novel pharmaceutical composition according to claim 24, wherein the ratio of biologically active agent: absorption enhancer lies in the range of 1:100 to 1:0.01 and preferably in the range of 1:10 to 1:0.1

37. A novel pharmaceutical composition according to claim 24, wherein the nanoparticles have particle size between 10-1000 nm.

38. A novel pharmaceutical composition according to claim 37, wherein the nanoparticles have particle size preferably less than 500 nm.

39. A novel pharmaceutical composition according to claim 24, which may be formulated as powders, sprays, suspensions, freeze dried powders for reconstitution, tablets, capsules, pellets, wafers, patches, films, rods, pessaries, suppositories, aerosols, gels or creams.

40. A novel pharmaceutical composition according to claim 24, optionally contains surfactants, preferably poloxamer.

41. A novel pharmaceutical composition according to claim 24, provides enhanced bioavailability of claimed biologically active agent when delivered by sublingual, buccal, gingival, nasal or rectal route.

42. A novel pharmaceutical composition for enhanced absorption of biologically active agent comprising of plurality of nanoparticles prepared from polymers, and at least one absorption enhancer as substantially described herein and illustrated in reference to the examples.
